(19) United States
(12) Patent Application Publication
(10) Pub. No.: US 2026/0347402 A1
(43) Pub. Date: Nov. 5, 2026

(54) Method for Generating Sequence of Antimicrobial Peptide and Antimicrobial Peptide

(71) Applicant: KAOHSIUNG MEDICAL UNIVERSITY, Kaohsiung (TW)

(72) Inventors: Po-Liang LU, Kaohsiung City (TW); Yeng-Tseng WANG, Tainan City (TW); Tsung-Ying YANG, Pingtung County (TW); Yu-Wei CHEN, Kaohsiung City (TW)

(21) Appl. No.: 19/660,602

(22) Filed: Apr. 28, 2026

Related U.S. Application Data
(60) Provisional application No. 63/797,126, filed on Apr. 30, 2025.

(51) Int. Cl. C07K 7/08; G16B 15/00

(52) U.S. Cl. CPC C07K 7/08; G16B 15/00

(57) ABSTRACT

A method for generating a sequence of an antimicrobial peptide is provided to solve the problem that conventional methods lack transparency and are therefore difficult to understand and adjust. The method comprises receiving a plurality of sets of antimicrobial peptide information from a database; calculating a corresponding isoelectric point and a helical structure ratio based on each set of the antimicrobial peptide information; statistically analyzing all of the isoelectric points and the helical structure ratios to establish a fitness function; optimizing an amino acid sequence based on the fitness function to generate a candidate sequence; and evaluating antimicrobial potential of the candidate sequence through an antimicrobial peptide index. An antimicrobial peptide having the amino acid sequence generated by the method is also provided.

### CROSS REFERENCE TO RELATED APPLICATION

The application claims the benefit of the U.S. provisional application Ser. No. 63/797,126, filed on Apr. 30, 2025, and the entire contents of which are incorporated herein by reference.

### STATEMENT REGARDING SEQUENCE LISTING

The sequence listing associated with this application is provided in text format in lieu of a paper copy and is hereby incorporated by reference into the specification. The name of the XML file containing the sequence listing is sl.xml. The XML file is 11,321 bytes; was created on May 14, 2026; and is being submitted electronically via Patent Center.

### BACKGROUND OF THE INVENTION

### 1. Field of the Invention

The present invention relates to a biological feature analysis and genetic algorithm method, and more particularly to a method for generating antimicrobial peptide sequences that achieves sequence optimization and generates sequences with potential antimicrobial activity. The present invention also relates to an antimicrobial peptide having an amino acid sequence generated by the method for generating antimicrobial peptide sequences.

### 2. Description of the Related Art

Antimicrobial Peptides (Antimicrobial Peptides, AMPs) possess diversity and antimicrobial capability, and are able to rapidly destroy bacterial cell membranes. They are widely applied in the medical field to combat Gram-positive bacteria, Gram-negative bacteria, and fungi, serving as potential alternatives to traditional antibiotics and addressing antibiotic resistance issues. The application of naturally sourced antimicrobial peptides is limited, requiring database comparison and experimental verification processes, with repeated trials and modification of antimicrobial peptide sequences to enhance antimicrobial capability. However, the generation of antimicrobial peptide sequences lacks systematic planning and optimization processes, which limits research and development efficiency.

Conventional methods for generating antimicrobial peptide sequences typically utilize deep learning training models, with artificial intelligence assisting in planning and screening antimicrobial peptides with high antimicrobial potential and low toxicity. However, the decision-making process of deep learning models is complex and difficult to understand. Typically, users can only select input information and receive output results, without considering the model structure and operational logic, and cannot make adjustments or provide explanations. The process of obtaining antimicrobial peptide sequences lacks transparency, which may lead to bias and reduced reliability.

In view of this, it is necessary to improve the conventional methods for generating antimicrobial peptide sequences.

### SUMMARY OF THE INVENTION

To solve the above problems, it is an objective of the present invention to provide a method for generating antimicrobial peptide sequences that can automatically generate peptide sequences with high antimicrobial potential.

It is another objective of the present invention to provide a method for generating antimicrobial peptide sequences that can dynamically adjust screening criteria.

It is yet another objective of the present invention to provide an antimicrobial peptide having an amino acid sequence generated by the method for generating antimicrobial peptide sequences.

As used herein, the term “a”, “an” or “one” for describing the number of the elements and members of the present invention is used for convenience, provides the general meaning of the scope of the present invention, and should be interpreted to include one or at least one. Furthermore, unless explicitly indicated otherwise, the concept of a single component also includes the case of plural components.

A method for generating antimicrobial peptide sequences according to the present invention comprises: receiving a plurality of sets of antimicrobial peptide information from a database; calculating a corresponding isoelectric point and a helical structure ratio based on each set of the antimicrobial peptide information; statistically analyzing all of the isoelectric points and the helical structure ratios to establish a fitness function; optimizing an amino acid sequence based on the fitness function to generate a candidate sequence; and evaluating antimicrobial potential of the candidate sequence through an antimicrobial peptide index.

Therefore, the method for generating antimicrobial peptide sequences of the present invention, by statistically analyzing antimicrobial peptide information and establishing a screening model, and dynamically adjusting screening criteria based on physiochemical characteristic indicators, can automatically generate peptide sequences with high antimicrobial potential, and has the effects of reducing computational complexity, improving reliability, and having adjustable parameters.

In an example, the database is the CAMPR3 antimicrobial peptide database. Thus, original and known sequences can be collected, achieving the effect of referencing and analyzing antimicrobial peptides.

In an example, the isoelectric point is calculated through industrial PC software; and protein secondary structure prediction software is used to predict a secondary structure of antimicrobial peptide molecules and calculate the helical structure ratio. Thus, antimicrobial peptide characteristics and properties can be analyzed, achieving the effect of researching and classifying antimicrobial peptides.

In an example, the fitness function includes a first threshold, with the helical structure ratio greater than 0.48; and a second threshold, with the isoelectric point greater than 9.63. Thus, antimicrobial peptides can be determined and classified, achieving the effect of selecting excellent antimicrobial peptides.

In an example, a third threshold of the fitness function is that the antimicrobial peptide index is lower than a set value, with the set value being 0.25. Thus, screening can be performed based on antimicrobial capability, achieving the effect of evaluating the antimicrobial potential.

In an example, the fitness function includes an additional condition, with a similarity between the antimicrobial peptide sequence and published antimicrobial peptide sequences of the United States Patent and Trademark Office database lower than or equal to 40%. Thus, known or similar antimicrobial peptide sequences can be screened out, achieving the effect of ensuring the generation of new sequences.

In an example, selection, crossover, and mutation are performed on the amino acid sequence using a genetic algorithm, new amino acid sequences are selected based on the antimicrobial peptide index, and the above steps are repeated until optimization is completed or repetition reaches 30 generations, to generate at least one candidate sequence. Thus, excellent sequences can be generated through iterative evolution, achieving the effect of optimizing antimicrobial peptide sequences.

In an example, weighted selection is performed based on the fitness function value, using a roulette wheel selection method to retain amino acid sequences with high scores. Thus, sequences that do not meet the conditions can be eliminated, achieving the effect of retaining sequences with high fitness.

In an example, a two-point crossover method is used to pair excellent amino acid sequences, with a pairing probability between 0.8~1. Thus, excellent portions can be retained for generating the next generation, achieving the effect of generating excellent new sequences.

In an example, the mutation is performed by randomly selecting two positions within the amino acid sequence for amino acid substitution, with a substitution probability less than 0.05. Thus, the limitations of the previous generation sequence can be broken through, achieving the effect of randomly generating optimal sequences.

In an example, the calculation of the antimicrobial peptide index is performed based on the amino acid index table compiled by Marc Torrent. Thus, the antimicrobial peptide coefficient value can be used to determine whether there is a chance of becoming an antimicrobial peptide, achieving the effect of screening sequences.

An antimicrobial peptide according to the present invention may comprise an amino acid sequence set forth in SEQ ID NO: 1. For example, the antimicrobial peptide may comprise an amino acid sequence set forth in SEQ ID NOS: 2~9. It has been experimentally confirmed that the antimicrobial peptide has inhibitory capability against microorganisms belonging to Enterobacteriaceae (such as E. coli, K. pneumoniae), Enterococcaceae (such as E. faecalis), Staphylococcaceae (such as S. aureus, S. epidermidis), Moraxellaceae (such as A. baumannii), and Pseudomonadaceae (such as P. aeruginosa).

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 shows a flow chart of a method of a preferred embodiment according to the present invention.

FIG. 2 shows an amino acid sequence alignment of the antimicrobial peptides set forth in SEQ ID NOS: 2-9. Alanine (A), Lysine (L), Tryptophan (W), Isoleucine (I), Methionine (M) and Phenylalanine (F) indicate positively charged basic amino acid residues; Lysine (K) and Arginine (R) indicate hydrophobic amino acid residues; Glycine (G), Histidine (H), Asparagine (N) and Glutamine (Q) indicate amino acid residues having neutral or polar side chains; and Glutamic acid (E) indicates negatively charged acidic amino acid residue. In addition, an asterisk (*) indicates positions of identical amino acid residues among the sequences, and a dash (-) indicates a gap inserted for sequence alignment.

### DETAILED DESCRIPTION OF THE INVENTION

In order to make the above and other objectives, features, and advantages of the present invention clearer and easier to understand, the preferred embodiments of the present invention will be described hereinafter in connection with the accompanying drawings. Furthermore, the elements designated by the same reference numeral in various figures will be deemed as identical, and the description thereof will be omitted.

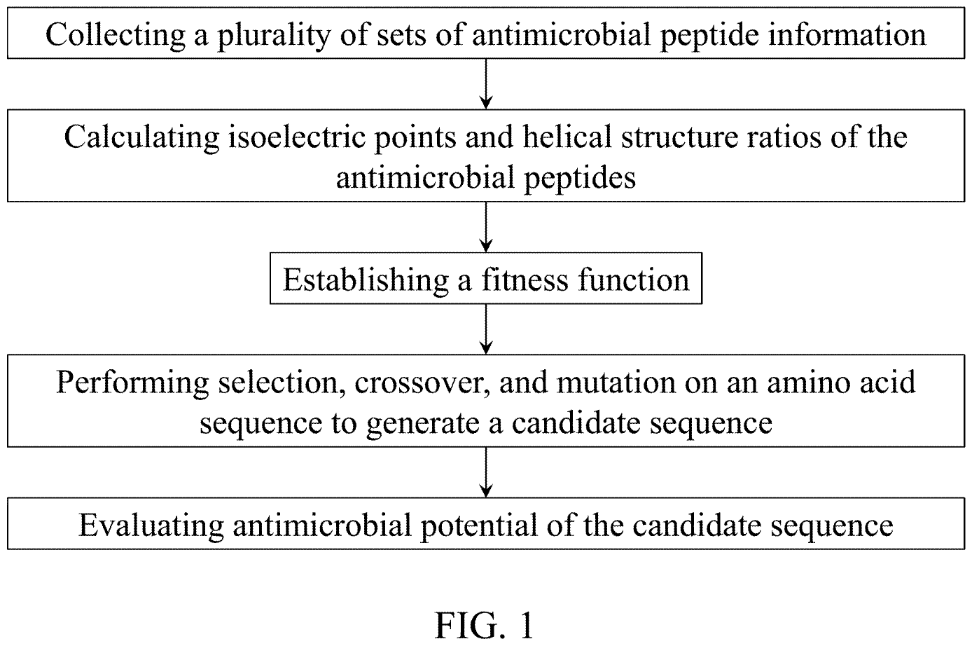

Please refer to FIG. 1, which shows a preferred embodiment of the method for generating antimicrobial peptide sequences of the present invention, comprising: collecting a plurality of sets of antimicrobial peptide information; calculating the isoelectric point (isoelectric point, pI) and helical structure ratio of the antimicrobial peptides; establishing a fitness function; performing selection, crossover, and mutation on an amino acid sequence to generate a candidate sequence; and evaluating the antimicrobial potential of the candidate sequence.

Each of the antimicrobial peptide information may include characteristics and physicochemical properties of various antimicrobial peptides such as sequence, source, net charge, and hydrophobicity. The plurality of sets of antimicrobial peptide information may be stored in a database for collecting, researching, and analyzing various antimicrobial peptides. In this embodiment, the database may be CAMPR3 (Collection Antimicrobial Peptides, version 3); however, the present invention is not limited thereto.

After collecting the plurality of sets of antimicrobial peptide information from the database, the corresponding isoelectric point and helical structure ratio can be calculated based on each of the antimicrobial peptide information. The isoelectric point is the pH value at which the antimicrobial peptide molecule carries no net charge or is electrically neutral. The isoelectric point can be calculated through industrial PC (IPC) software. Furthermore, the secondary structure of antimicrobial peptide molecules includes structures such as helix, sheet, and coil. The helical structure ratio is the proportion of the helical structure of the antimicrobial peptide molecule among all secondary structures. Protein secondary structure predictor (PSIPRED) software can be used to predict the secondary structure of antimicrobial peptide molecules and calculate the helical structure ratio.

All calculated isoelectric points and helical structure ratios are statistically analyzed to establish a fitness function. The fitness function can be used to determine and classify antimicrobial peptides. Specifically, the fitness function may include several determination conditions, such as: a first threshold, in which the helical structure ratio is greater than 0.48; a second threshold, in which the isoelectric point is greater than 9.63; a third threshold, in which an antimicrobial peptide index is lower than a set value, with the set value determined based on the application requirements of the antimicrobial peptide, and with the set value being 0.25. In addition, the fitness function may also include an additional condition, in which the similarity between the antimicrobial peptide sequence and the published antimicrobial peptide sequences in the United States Patent and Trademark Office database is lower than or equal to 40%.

Optimizing the amino acid sequence to be evolved based on the fitness function may be performed by using a genetic algorithm (GA) to evolve the amino acid sequence. Specifically, the genetic algorithm includes the following steps. Selection: weighted selection is performed based on the fitness function value, using a roulette wheel selection method to retain amino acid sequences with high scores. Crossover: a two-point crossover method is used to pair excellent amino acid sequences, with a pairing probability between 0.8~1, preferably 0.9. Mutation: randomly selecting two positions within the amino acid sequence for amino acid substitution, with a substitution probability less than 0.05, preferably 0.03. Reproduction: new amino acid sequences are selected based on the antimicrobial peptide index, and if the average fitness does not meet the standard, the above steps are repeated to continue the next generation of evolution, until the population fitness value stabilizes or repetition reaches 30 generations, to generate at least one candidate sequence.

The antimicrobial potential of the candidate sequence can be evaluated through the antimicrobial peptide index, selecting the candidate sequence with the antimicrobial peptide index lower than the set value. The antimicrobial peptide index can be based on statistical amino acid distribution and individual amino acid correlation index. The smaller the antimicrobial peptide index, the easier it is to be absorbed and the less likely it is to develop drug resistance. The top ten candidate sequences with the best indicators can be output as potential antimicrobial peptide sequences. The calculation of the antimicrobial peptide index can be performed based on the amino acid index table compiled by Marc Torrent.

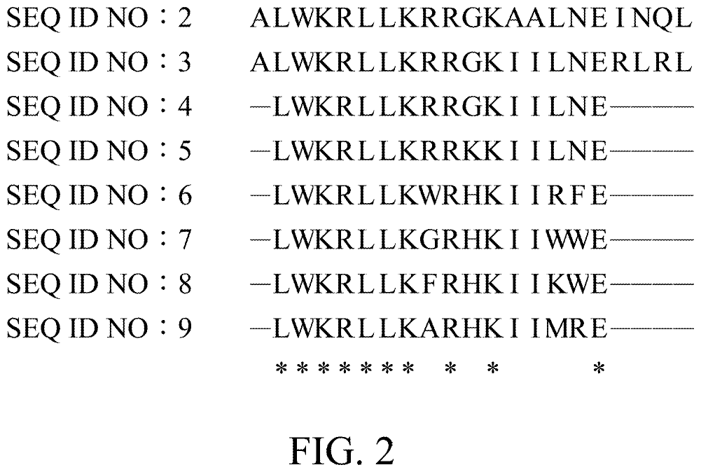

The peptides predicted by the foregoing method may comprise the amino acid sequence represented by the following general formula (SEQ ID NO: 1), or may consist of the amino acid sequence represented by SEQ ID NO: 1. In one embodiment, the general formula is LWKRLLKX₁RX₂KX₃X₄X₅X₆E, wherein X₁ may be Arginine (R), Tryptophan (W), Glycine (G), Phenylalanine (F), or Alanine (A); X₂ may be Glycine (G), Lysine (K), or Histidine (H); X₃ may be Alanine (A) or Isoleucine (I); X₄ may be Alanine (A) or Isoleucine (I); X₅ may be Lysine (L), Arginine (R), Tryptophan (W), Lysine (K), or Methionine (M); and X₆ may be Asparagine (N), Phenylalanine (F), Tryptophan (W), or Arginine (R). By selecting the foregoing variable positions, a number of antimicrobial peptides having different amino acid sequence compositions may be formed. For example, the antimicrobial peptide may comprise the amino acid sequence set forth in SEQ ID NO: 2 (ALWKRLLKRRGKAALNEINQL), the amino acid sequence set forth in SEQ ID NO: 3 (ALWKRLLKRRGKIILNERLRL), the amino acid sequence set forth in SEQ ID NO: 4 (LWKRLLKRRGKIILNE), the amino acid sequence set forth in SEQ ID NO: 5 (LWKRLLKRRKKIILNE), the amino acid sequence set forth in SEQ ID NO: 6 (LWKRLLKWRHIIRFE), the amino acid sequence set forth in SEQ ID NO: 7 (LWKRLLKGRHKIIWWE), the amino acid sequence set forth in SEQ ID NO: 8 (LWKRLLKFRHKIIKWE), or the amino acid sequence set forth in SEQ ID NO: 9 (LWKRLLKARHKIIMRE), or may consist of any one of the amino acid sequences set forth in the foregoing SEQ ID NOS: 2-9. The sequence alignment results of the peptides having the amino acid sequences set forth in SEQ ID NOS: 2-9 are shown in FIG. 2.

The foregoing antimicrobial peptides may be prepared by chemical synthesis. For example, when the antimicrobial peptide is a short-chain peptide consisting only of 16 to 21 amino acid residues, solid-phase peptide synthesis (SPPS) may be employed to sequentially couple the respective amino acid residues onto a solid support so as to form a short peptide having the corresponding target amino acid sequence. Thereafter, deprotection, cleavage, purification, desalting, and other treatments may be performed as needed to obtain the antimicrobial peptide.

Alternatively, a person having ordinary skill in the art may design a nucleic acid sequence encoding the foregoing antimicrobial peptide according to the amino acid sequence thereof, and construct the nucleic acid sequence into an expression vector to form a corresponding recombinant construct, and then introduce the recombinant construct into a host cell for expression. The foregoing nucleic acid sequence may be subjected to codon optimization according to the intended host cell, that is, codon usage suitable for the host cell may be employed to improve expression efficiency of the antimicrobial peptide. The host cell may be a prokaryotic host cell or a eukaryotic host cell, such as an Escherichia coli cell, a yeast cell, an insect cell, or a mammalian cell. The expressed product may also be subjected to cell lysis, chromatographic separation, removal of a fusion tag, purification, and the like, so as to obtain an antimicrobial peptide having the corresponding target amino acid sequence. In order to improve expression level, stability, or convenience of purification of the antimicrobial peptide, a person having ordinary skill in the art may also choose to first express the antimicrobial peptide in the form of a fusion protein, followed by release of the antimicrobial peptide by enzymatic cleavage, chemical cleavage, or the like.

In addition, a person having ordinary skill in the art may further formulate the antimicrobial peptide into various suitable pharmaceutical or formulation compositions according to actual needs, so as to facilitate storage, transport, administration, or improve stability, bioavailability, and efficacy thereof. For example, the antimicrobial peptide may be formulated into various forms of formulation compositions, such as a solution, suspension, emulsion, colloid, microparticle, nanoparticle, liposome, gel, powder, or film, as would be understood by a person having ordinary skill in the art, and thus is not limiting herein. The foregoing formulation compositions may further be administered, according to actual application needs, by oral administration, injection, external use, coating, dropping, spraying, or other suitable means.

In one embodiment, the antimicrobial peptide may be formulated as a liposomal formulation. For example, the liposomal formulation may be prepared by a thin-film hydration technique. Specifically, a person having ordinary skill in the art may mix lipid materials such as 1,2-dioleoyl-sn-glycero-3-phosphocholine (DOPC) and 1,2-distearoyl-sn-glycero-3-phosphoethanolamine-N-[polyethylene glycol 2000](DSPE-PEG2000) at a ratio of about 1:1, dissolve the mixture in an organic solvent such as chloroform, and then remove the chloroform by rotary evaporation under reduced pressure, thereby forming a lipid film. Next, an aqueous antimicrobial peptide solution (dissolved in deionized water) is added into a round-bottom flask containing the lipid film, and after vortex mixing, the lipid film is hydrated to form a liposomal suspension. After allowing the liposomal suspension to stand for a period of time, centrifugation is performed to separate the supernatant, and the resulting precipitate is resuspended in deionized water and vortexed again, thereby forming a redispersed liposomal suspension.

Further, in order to promote formation of liposomal nanoparticles, the redispersed liposomal suspension may be further combined with an aqueous solution containing poly-L-lysine (PLL), and allowed to stand at room temperature for a period of time, thereby forming liposomal nanoparticles comprising the antimicrobial peptide. However, the foregoing preparation process for the liposomal formulation is merely exemplary. A person having ordinary skill in the art may also prepare the liposomal formulation by other methods, such as a reverse-phase evaporation method, an injection method, or a microfluidic preparation method, and may also adjust particle size, surface properties, lipid composition, or types of excipients according to actual use. The present invention is not limited thereto.

In order to evaluate the antimicrobial activity of the respective antimicrobial peptides, each antimicrobial peptide was prepared with water into a peptide solution having an initial concentration (the concentration being 4096 μg/ml), followed by 2-fold serial dilution to form multiple test samples having different concentrations.

Next, the test samples of different concentrations were respectively mixed with the test microorganisms (prepared as bacterial suspensions having a concentration of 7.5×10⁵ CFU/mL), and the mixed samples were dispensed into a 96-well plate and cultured at 37 °C for 16-18 hours. The growth of each test microorganism at each concentration was observed, and the lowest peptide concentration capable of completely inhibiting microbial growth was determined as the minimum inhibitory concentration (MIC) of the antimicrobial peptide against the test microorganism. The results are shown in TABLE 1.

TABLE 1
Minimum inhibitory concentrations of the
antimicrobial peptides against test microorganisms

| Antimi- | Minimum inhibitory concentration (μg/mL) | | | | | | |
|---|---|---|---|---|---|---|---|
| crobial | | K. | E | S. | S. | A | P. |
| peptides | E. coli | pneumoniae | faecalis | aureus | epidermidis | baumannii | aeruginosa |
| SEQ | 1024 | 1024 | 1024 | 512 | 16 | 64 | 64 |
| ID | | | | | | | |
| NO: 2 | | | | | | | |
| SEQ | 64 | 128 | 64 | 64 | 2 | 8 | 8 |
| ID | | | | | | | |
| NO: 3 | | | | | | | |
| SEQ | 512 | 512 | 512 | 128 | 8 | 64 | 64 |
| ID | | | | | | | |
| NO: 4 | | | | | | | |
| SEQ | 512 | 512 | 256 | 64 | 8 | 64 | 512 |
| ID | | | | | | | |
| NO: 5 | | | | | | | |
| SEQ | 128 | 128 | 128 | 128 | 32 | 128 | 8 |
| ID | | | | | | | |
| NO: 6 | | | | | | | |
| SEQ | 128 | 128 | 128 | 128 | 8 | 2 | 4 |
| ID | | | | | | | |
| NO: 7 | | | | | | | |
| SEQ | 128 | 128 | 128 | 32 | 4 | 4 | 2 |
| ID | | | | | | | |
| NO: 8 | | | | | | | |
| SEQ | 128 | 64 | 64 | 64 | 4 | 4 | 4 |
| ID | | | | | | | |
| NO: 9 | | | | | | | |

As shown in TABLE 1, the antimicrobial peptides all exhibited inhibitory activity against microorganisms belonging to the family Enterobacteriaceae (e.g., E. coli and K. pneumoniae), the family Enterococcaceae (e.g., E. faecalis), the family Staphylococcaceae (e.g., S. aureus and S. epidermidis), the family Moraxellaceae (e.g., A. baumannii), and the family Pseudomonadaceae (e.g., P. aeruginosa), with minimum inhibitory concentrations ranging from 2 to 1024 μg/mL. Among the tested antimicrobial peptides, the peptide having the amino acid sequence set forth in SEQ ID NO: 3 (ALWKRLLKRRGKIILNERLRL) exhibited better antimicrobial activity.

In summary, the method for generating antimicrobial peptide sequences of the present invention, by statistically analyzing antimicrobial peptide information and establishing a screening model, and dynamically adjusting screening criteria based on physiochemical characteristic indicators, can automatically generate peptide sequences with high antimicrobial potential, and has the effects of reducing computational complexity, improving reliability, and having adjustable parameters.

Furthermore, it has been experimentally confirmed that the antimicrobial peptide has inhibitory capability against microorganisms belonging to Enterobacteriaceae (such as E. coli, K. pneumoniae), Enterococcaceae (such as E. faecalis), Staphylococcaceae (such as S. aureus, S. epidermidis), Moraxellaceae (such as A. baumannii), and Pseudomonadaceae (such as P. aeruginosa).

Although the present invention has been described with respect to the above preferred embodiments, these embodiments are not intended to restrict the present invention. Various changes and modifications on the above embodiments made by any person skilled in the art without departing from the spirit and scope of the present invention are still within the technical category protected by the present invention. Accordingly, the scope of the present invention shall include the literal meaning set forth in the appended claims and all changes which come within the range of equivalency of the claims. Furthermore, in a case that several of the above embodiments can be combined, the present invention includes the implementation of any combination.

What is claimed is:

1. A method for generating antimicrobial peptide sequences, comprising:
receiving a plurality of sets of antimicrobial peptide information from a database;
calculating a corresponding isoelectric point and a helical structure ratio based on each set of the antimicrobial peptide information;
statistically analyzing all of the isoelectric points and the helical structure ratios to establish a fitness function;
optimizing an amino acid sequence based on the fitness function to generate a candidate sequence; and
evaluating antimicrobial potential of the candidate sequence through an antimicrobial peptide index.

2. The method for generating antimicrobial peptide sequences as claimed in claim 1, wherein the database is the CAMPR3 antimicrobial peptide database.

3. The method for generating antimicrobial peptide sequences as claimed in claim 1, wherein the isoelectric point is calculated through industrial PC software; and protein secondary structure prediction software is used to predict a secondary structure of antimicrobial peptide molecules and calculate the helical structure ratio.

4. The method for generating antimicrobial peptide sequences as claimed in claim 1, wherein the fitness function includes a first threshold, wherein the helical structure ratio is greater than 0.48; and a second threshold, wherein the isoelectric point is greater than 9.63.

5. The method for generating antimicrobial peptide sequences as claimed in claim 4, wherein a third threshold of the fitness function is that the antimicrobial peptide index is lower than a set value, wherein the set value is 0.25.

6. The method for generating antimicrobial peptide sequences as claimed in claim 4, wherein the fitness function includes an additional condition, wherein a similarity between the antimicrobial peptide sequence and published antimicrobial peptide sequences of the United States Patent and Trademark Office database is lower than or equal to 40%.

7. The method for generating antimicrobial peptide sequences as claimed in claim 1, wherein selection, crossover, and mutation are performed on the amino acid sequence using a genetic algorithm, new amino acid sequences are selected based on the antimicrobial peptide index, and the above steps are repeated until optimization is completed or repetition reaches 30 generations, to generate at least one candidate sequence.

8. The method for generating antimicrobial peptide sequences as claimed in claim 7, wherein weighted selection is performed based on the fitness function value, using a roulette wheel selection method to retain amino acid sequences with high scores.

9. The method for generating antimicrobial peptide sequences as claimed in claim 7, wherein a two-point crossover method is used to pair excellent amino acid sequences, with a pairing probability between 0.8~1.

10. The method for generating antimicrobial peptide sequences as claimed in claim 7, wherein the mutation is performed by randomly selecting two positions within the amino acid sequence for amino acid substitution, with a substitution probability less than 0.05.

11. The method for generating antimicrobial peptide sequences as claimed in claim 1, wherein the calculation of the antimicrobial peptide index is performed based on the amino acid index table compiled by Marc Torrent.

12. An antimicrobial peptide comprising an amino acid sequence set forth in SEQ ID NO: 1.

13. The antimicrobial peptide as claimed in claim 12, wherein the antimicrobial peptide comprising an amino acid sequence set forth in SEQ ID NO: 2.

14. The antimicrobial peptide as claimed in claim 13, wherein the antimicrobial peptide comprising an amino acid sequence set forth in SEQ ID NO: 3.

15. The antimicrobial peptide as claimed in claim 14, wherein the antimicrobial peptide comprising an amino acid sequence set forth in SEQ ID NO: 4.

16. The antimicrobial peptide as claimed in claim 15, wherein the antimicrobial peptide comprising an amino acid sequence set forth in SEQ ID NO: 5.

17. The antimicrobial peptide as claimed in claim 16, wherein the antimicrobial peptide comprising an amino acid sequence set forth in SEQ ID NO: 6.

18. The antimicrobial peptide as claimed in claim 17, wherein the antimicrobial peptide comprising an amino acid sequence set forth in SEQ ID NO: 7.

19. The antimicrobial peptide as claimed in claim 18, wherein the antimicrobial peptide comprising an amino acid sequence set forth in SEQ ID NO: 8.

20. The antimicrobial peptide as claimed in claim 19, wherein the antimicrobial peptide comprising an amino acid sequence set forth in SEQ ID NO: 9.


SEQUENCE LISTING (9 sequences)

SEQ ID NO: 1 - protein, 16 aa, synthetic construct
  VARIANT 8: note = Arginine, Tryptophan, Glycine, Phenylalanine or Alanine
  VARIANT 10: note = Glycine, Lysine or Histidine
  VARIANT 12: note = Alanine or Isoleucine
  VARIANT 13: note = Alanine or Isoleucine
  VARIANT 14: note = Leucine, Arginine, Tryptophan, Lysine or Methionine
  VARIANT 15: note = Asparagine, Phenylalanine, Tryptophan or Arginine
LWKRLLKXRXKXXXXE

SEQ ID NO: 2 - protein, 21 aa, synthetic construct
ALWKRLLKRRGKAALNEINQL

SEQ ID NO: 3 - protein, 21 aa, synthetic construct
ALWKRLLKRRGKIILNERLRL

SEQ ID NO: 4 - protein, 16 aa, synthetic construct
LWKRLLKRRGKIILNE

SEQ ID NO: 5 - protein, 16 aa, synthetic construct
LWKRLLKRRKKIILNE

SEQ ID NO: 6 - protein, 16 aa, synthetic construct
LWKRLLKWRHKIIRFE

SEQ ID NO: 7 - protein, 16 aa, synthetic construct
LWKRLLKGRHKIIWWE

SEQ ID NO: 8 - protein, 16 aa, synthetic construct
LWKRLLKFRHKIIKWE

SEQ ID NO: 9 - protein, 16 aa, synthetic construct
LWKRLLKARHKIIMRE